(19) United States
(12) Patent Application Publication
(10) Pub. No.: US 2022/0265399 A1
(43) Pub. Date: Aug. 25, 2022

(54) SYSTEM, METHOD, AND COMPUTER PROGRAMS FOR THE PLACEMENT OF DENTAL IMPLANTS

(71) Applicant: ACCURATE FIT, SL, Sevilla (ES)

(72) Inventor: Angel CALLE BONET, Sant Cugat del Valles (ES)

(73) Assignee: ACCURATE FIT, SL, Sevilla (ES)

(21) Appl. No.: 17/628,315

(22) PCT Filed: Jul. 14, 2020
(86) PCT No.: PCT/EP2020/069862

§ 371 Date: Jan. 19, 2022

(30) Foreign Application Priority Data
Jul. 23, 2019 (EP) 19000341.8
Aug. 23, 2019 (ES) P201930755

(51) Int. Cl. A61C 8/00; A61B 90/00; A61C 9/00; A61B 6/14; A61B 6/03; A61B 6/00

(52) U.S. Cl. CPC A61C 8/0089; A61B 90/39; A61C 9/0046; A61B 6/14; A61B 6/032; A61B 6/4085; A61B 2090/3966; A61B 2090/3912

(57) ABSTRACT

The present invention relates to a system, method, and computer programs for the placement of dental implants. The system comprises posts for modeling dental implants, each for the coupling thereof in a dental implant of a patient, wherein each post includes a plurality of first locators made of a radiologically visible material; a reference element suitable for the positioning thereof around the teeth of said patient, wherein said reference element includes a series of second locators made of a radiologically visible material, distributed at prefixed distance and position on a surface of the reference element; an image acquisition system for obtaining a three-dimensional image of the posts and of said reference element; and a processing unit configured for processing said three-dimensional image acquired by means of implementing algorithms calculating the position and axial orientation of each of the posts present in the image, providing a file.

### FIELD OF THE INVENTION

The present invention generally relates to the field of dental implants. In particular, the invention relates to a system, method, and computer program for the high accuracy placement of dental implants.

### BACKGROUND OF THE INVENTION

European patent EP2907474-B1 describes a post for modeling dental implants by means of a radiologic test, said modeling comprising positioning X-ray detectable posts on the implants fitted in the mouth of a patient, performing a radiologic test of the patient's mouth having the posts positioned on the implants, converting the CT scan results into a three-dimensional computer model for CAD/CAM processing, defining the posts in the computer model, CAD/CAM modeling of the structure according to the computer model with the defined posts, and generating a file with the positions and orientations for calculation of the milling program for the dental prosthesis structures.

Patent EP1722710-B1 describes a method and marker element for determining the position of a dental implant which, like in the aforementioned prior art document, is based on the use of a single marker element which is fixed to a free end of said implant and produces a contrast in the X-ray or magnetic resonance imaging techniques.

WO2017070358-A1 discloses an attachment member for mating with a dental implant that includes a non-rotational structure and a body. The non-rotational structure is configured to mate with a corresponding non-rotational feature of the dental implant. The body extends from the non-rotational structure. The body has (i) an exterior side surface configured to at least partially engage gingival tissue adjacent to the dental implant, (ii) an exterior top surface that is exposed through the gingival tissue, (iii) a screw access bore for receiving a screw that attaches the attachment member to the dental implant in a removable fashion, and (iv) a set of radiopaque information markers that is located internal to the exterior side surface and the exterior top surface. The set of radiopaque information markers indicates information regarding the dental implant that is revealed in response to a scan from a computerized tomography (CT) scanner.

U.S. Pat. No. 7,751,865-B2 discloses a surgical navigation system for navigating a region of a patient that may include a non-invasive dynamic reference frame and/or fiducial marker, sensor tipped instruments, and isolator circuits. The dynamic reference frame may be placed on the patient in a precise location for guiding the instruments. The dynamic reference frames may be fixedly placed on the patient. Also the dynamic reference frames may be placed to allow generally natural movements of soft tissue relative to the dynamic reference frames. Also methods are provided to determine positions of the dynamic reference frames. Anatomical landmarks may be determined intra-operatively and without access to the anatomical structure.

US2005163342-A1 relates to method for correcting inherent distortions in a CT or MRI imaging process, or distortions arising from excessive patient movement during the scan by means of a registration device inserted into the mouth of the patient at the time the scan is being performed. The registration device incorporates a set of fiducial markers disposed in a predetermined three-dimensional pattern. The exact positions of the fiducial markers are known with respect to each other, thus providing a three-dimensional reference against which the resulting images can be compared.

Moreover, scientific articles [1, 2, 3] describe the existence of geometrical distortions in dental scanners. More specifically, scientific article [1] discloses a methodology for analyzing distortions in dental images obtained through cone-beam computed tomography techniques (CBCT). Scientific article [2] discloses a study comparing the accuracy of cone-beam computed tomography and computed tomography in the context of evaluating the diagnostic value and accuracy of fiducial marker localization for reference marker-based guided surgery systems. Scientific article [3] discloses another study for determining the reproducibility and accuracy of linear measurements in dental models derived from cone-beam computed tomography compared with digital dental casts.

Therefore, although the known prior art documents allow determining the orientation and position of dental implants with certain accuracy or compensating or correcting images obtained from a subject's teeth, new systems and methods for allowing an even more accurate placement of dental implants by means of a more accurate detection of reference or fiducial markers are required.

### REFERENCES

[1] John W. Ballrick et al. “Image distortion and spatial resolution of a commercially available cone-beam computed tomography machine”. 2008. American Association of Orthodontists.
[2] Marcus Abboud et al. “Comparison of the Accuracy of Cone-beam Computed Tomography and Medical Computed Tomography: Implications for Clinical Diagnostics with Guided Surgery”. Quintessence Publishing Co Inc. 536 Volume 28, Number 2, 2013.
[3] Olivier de Waard et al. “Reproducibility and accuracy of linear measurements on dental models derived from cone-beam computed tomography compared with digital dental casts”. 2014. American Association of Orthodontists.

### DESCRIPTION OF THE INVENTION

In order to solve the aforementioned drawbacks, the present invention provides, according to a first aspect, a system for the placement of dental implants, which comprises: one or more posts each for the coupling thereof in a dental implant of a patient, wherein each post includes a plurality of first locators made of a radiologically visible material; a reference element, adapted for the positioning thereof around the teeth of said patient, wherein said reference element includes a series of second locators made of a radiologically visible material, distributed at known (preferably with highly accurate measurements) prefixed distance and position on a surface of the reference element; an image acquisition system for obtaining at least one three-dimensional image of the posts and of the reference element when the posts with their first locators are coupled in the implant and when the reference element with its second locators is positioned around the teeth of the patient; and a processing unit.

According to the proposed system, the mentioned processing unit is configured for processing the at least one acquired three-dimensional image by means of implementing a first algorithm which detects how the second locators in the reference element are arranged in the image, calculates the distance between the second locators in the image, and calculates a correction factor by comparing said calculated distance with said known prefixed distance and position.

Likewise, the processing unit implements a second algorithm which detects in the image the first locators, groups them in correspondence to each post, by the number of elements and by proximity, applies an adjustment factor, taking into account said calculated correction factor, to one or more of the groups using a third point iteration algorithm, for example the ICP algorithm, providing a series of geometrical transformations of the first locators, and prepares a file using said series of geometrical transformations, such that the placement and axial orientation are indicated for each post.

The present invention is particularly useful for manufacturing bars for the replacement of multiple teeth by means of multiple prostheses.

In one embodiment, the first locators made of a radiologically visible material (also known as reference or fiducial markers) comprise one or more radiopaque markers and are preferably arranged in a specific pattern. Furthermore, the post/posts comprise a radiotransparent body which houses encapsulated therein first locators.

In one embodiment, the three-dimensional image is a tomographic image, for example, obtained by means of a cone-beam computed tomography technique, computed axial tomography technique, or the like.

In another embodiment, the mentioned reference element has a horseshoe shape or curve-concave wall, oriented towards the mouth of the patient, and is made particularly of a plastic material. The reference element may be suitable for the placement thereof inside the mouth of the patient or can include means/elements for the securing thereof on the outside of the mouth of the patient.

In yet another embodiment, the proposed system further includes a control element, preferably a ruby ball having known dimensions. The control element is suitable for the coupling/securing thereof to the patient during image acquisition.

According to a second aspect, embodiments of the present invention also provide a method for the placement of dental implants. The proposed method comprises acquiring, by means of an image acquisition system, at least one three-dimensional image of first locators made of a radiologically visible material included in one or more posts and of a reference element when each of said one or more posts is coupled in a dental implant of a patient and when the reference element is positioned around the teeth of the patient, wherein said reference element includes a series of second locators made of a radiologically visible material, distributed at prefixed distance and position on a surface of the reference element; and processing, by means of a processing unit, the acquired three-dimensional image.

The mentioned processing comprises implementing a series of algorithms to calculate the position and orientation of each of the posts present in the acquired image, sending this information in a file for subsequently designing multiprosthetic bars. To that end, more particularly the mentioned processing of the acquired image comprises implementing a first algorithm which detects how the second locators in the reference element are arranged in the image, calculates the distance between the second locators in the image, and calculates a correction factor by comparing said calculated distance with said prefixed distance and position; and implementing a second algorithm which detects in the image the first locators, groups the detected first locators, in correspondence to each post, by the number of elements and by proximity, applies an adjustment factor, taking into account said calculated correction factor, to one or more of the groups using a third point iteration algorithm, providing a series of geometrical transformations of the first locators, and prepares the mentioned file using said series of geometrical transformations, such that the placement and axial orientation are indicated for each post.

Particularly, in the proposed method, before detecting the arrangement of the second locators in the reference element, the first algorithm checks whether or not the image has experienced movement during the acquisition by using a control object having known dimensions, coupled/secured to the patient during the acquisition, and comparing the characteristics of the acquired image with those of the control object. If the result of the movement check certifies that the image has experienced movement, it is discarded and not taken into account for subsequent processing, acquiring a new image.

The mentioned comparison particularly comprises checking intensity characteristics, for example, the brightness or grayscale level, and morphological characteristics, for example, the diameter and sphericity, of the objects included in the image with the control object.

Other embodiments of the invention herein disclosed also include computer program products for performing the steps and operations of the method of the second aspect of the invention. More particularly, a computer program product is an embodiment having a computer-readable medium including computer program instructions coded therein which, when implemented in at least one processor of a computer system, cause the processor to perform the operations herein indicated as embodiments of the invention.

### BRIEF DESCRIPTION OF THE DRAWINGS

The foregoing and other features and advantages will be better understood based on the following detailed description of merely illustrative, non-limiting embodiments in reference to the attached drawings, in which:

FIGS. 1A and 1B show different views of an embodiment of a post with the first locators, or fiducial markers.

FIGS. 2A and 2B show different embodiments of the reference element provided by the present invention.

FIG. 3 is a flow chart of the steps that are performed for processing the acquired three-dimensional images of the post and reference element, according to an embodiment of the present invention.

### DETAILED DESCRIPTION OF SEVERAL EMBODIMENTS

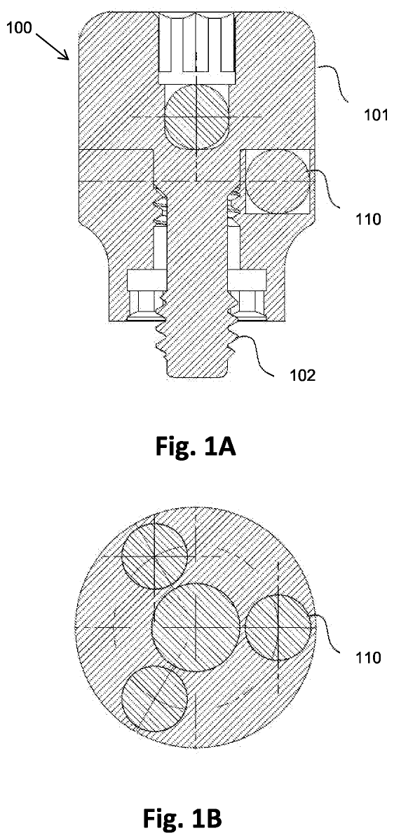

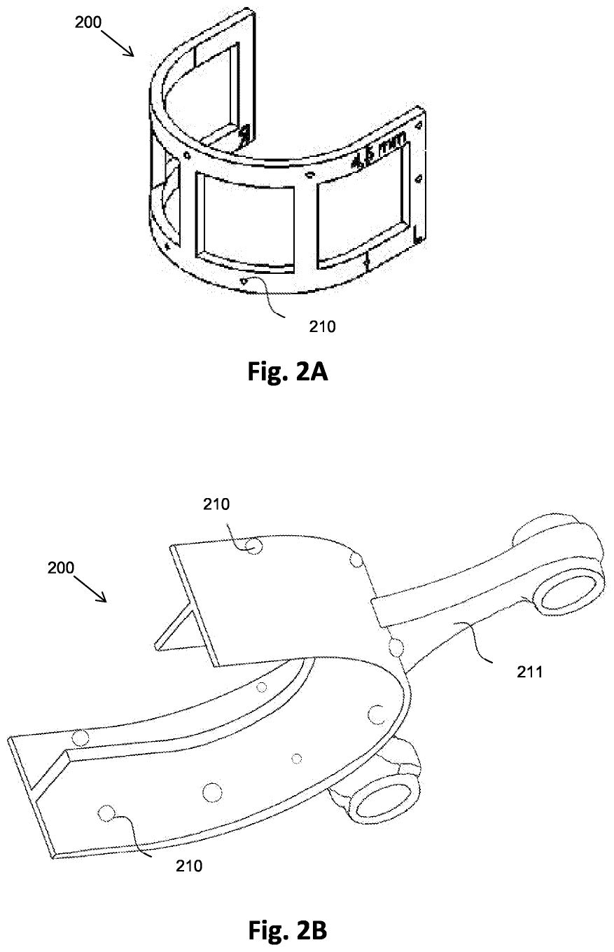

FIGS. 1 and 2A and 2B show, respectively, embodiments of a post 100 and of the reference element 200 of the proposed system.

As observed in FIGS. 1A and 1B, each post 100 comprises a radiotransparent body 101 with a threaded bolt 102 for the coupling thereof in a dental implant of a patient. Each post 100 internally defines a cavity in which there are housed fiducial markers 110 (or first locators as indicated in the claims) made of a material that is radiologically visible, for example, through a CT (computed tomography)-type ionizing radiation, CBCT (cone-beam computed tomography)-type ionizing radiation, or any other similar radiologic test. In the particular example shown in FIG. 1B, each post 100 includes three first locators 110 arranged in a specific pattern (which is non-limiting because it may include a larger number of locators). For example, the first locators 110 can be small ruby balls, all of them having one and the same or different diameter. In a particular embodiment, the each post 100 comprises 5 first locators 110. In other embodiments, the number of first locators 110 can be different.

The mentioned reference element 200 is suitable for the positioning thereof around the teeth of the patient, either inside the mouth or on the outside. If the reference element 200 is positioned outside the mouth, it includes securing means/elements for that purpose.

According to the example of FIG. 2A, the reference element 200 has a horseshoe shape or curved-concave surface and is made of a plastic material. The reference element 200 includes in turn a series of second locators 210 made of a radiologically visible material, distributed at known prefixed distance and position, on a surface of the reference element 200. The second locators 210 are made of the same material as the first locators 110 and have a similar or even larger shape. In a particular embodiment, the reference element 200 comprises between 10 and 14 second locators 210, particularly 12. In other embodiments, the number of second locators 210 can be different.

The reference element 200 of FIG. 2B additionally to the mentioned features also has grips or clamps 211 for attaching the reference element 200 to an image acquisition system.

The proposed system further includes a standard image acquisition system (not illustrated), for example a scanner, for obtaining one or more three-dimensional tomographic images of the posts 100 and of the reference element 200 when they are positioned in the patient. Likewise, the system incorporates a computing system (also not illustrated) with one or more processors and a memory for processing of the acquired image/images.

The proposed system also particularly includes a control element, more particularly a ruby ball having known dimensions, for the coupling/fastening thereof to the patient during image acquisition. The control element is used to check if the acquired image/images has/have experienced movement during the acquisition.

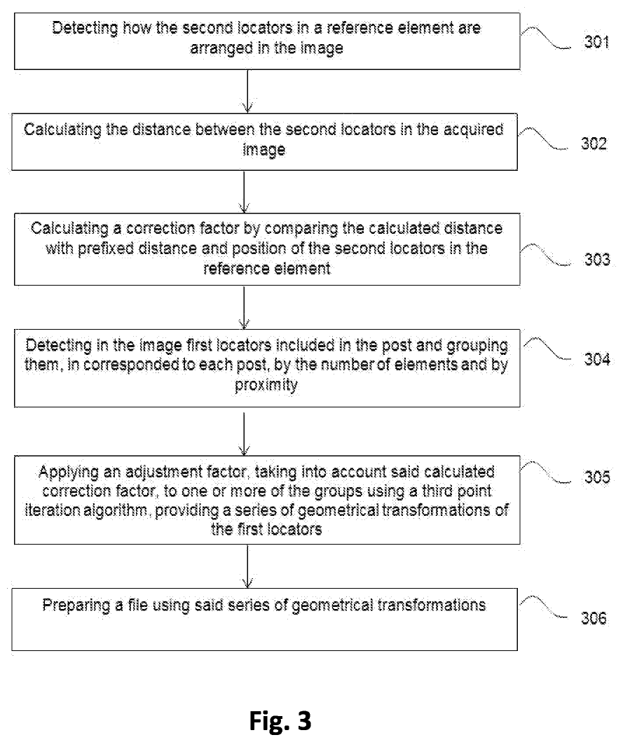

Now with reference to FIG. 3, it is shown an embodiment of the processing of an acquired three-dimensional image by the mentioned image acquisition system. The processing firstly includes, step 301, the detection, via a first algorithm or calibration algorithm, of how second locators in the mentioned reference element 200 are arranged in the acquired image, and the calculation, step 302, of the distance of the second locators in the acquired image. Subsequently, in step 303, a correction factor is calculated by comparing said calculated distance with the prefixed distance and position of the second locators on the surface of the reference element 200. Once the correction factor is calculated, a second algorithm or localization algorithm, step 304, detects in the image the first locators and groups them, in correspondence to each post 100, by the number of elements and by proximity. Then, in step 305, an adjustment factor is applied, taking into account said calculated correction factor, to one or more of the groups using a third point iteration algorithm, providing a series of geometrical transformations of the first locators. Finally, a file (for example, STL format or the like) is prepared using said series of geometrical transformations, such that the placement and axial orientation are indicated for each post 100.

In one embodiment, the mentioned processing also includes, prior to performing step 301, checking if the image has experienced movement during the acquisition (if this is the case, the image is not considered valid for subsequent processing, the image being discarded). To that end, upon receiving the image, a series of image processing algorithms which filter the elements according to their intensity characteristics (for example, brightness or grayscale level) and morphological characteristics (for example, diameter and sphericity) is applied, in search of an element in the image which complies with all the characteristics of the control object. If a single element is found, the image is labeled as suitable. In all the other cases, i.e., if more than one element and when no element is found, the image is labeled as unsuitable. The invention assumes that the movements of the patient during the acquisition introduce artifacts altering both the brightness and the shape of the control object in the acquired image. The control object therefore appears in the image with levels of brightness and shape that do not correspond with those that would be observed in a movement-free acquisition.

The proposed invention can be implemented in hardware, software, firmware, or any combination thereof. If it is implemented in software, the functions can be stored in or coded as one or more instructions or code in a computer-readable medium.

The computer-readable medium includes a computer storage medium. The storage medium can be any available medium that can be accessed by means of a computer. By way of non-limiting example, such computer-readable medium may comprise RAM, ROM, EEPROM, CD-ROM, or another optical disc storage, magnetic disk storage, or other magnetic storage devices, or any other medium that can be used for supporting or storing the desired computer code in the form of instructions or data structures and can be accessed by means of a computer. Disk and disc, as used herein, include compact discs (CDs), laser disc, optical disc, digital versatile disc (DVD), flexible disk, and Blu-ray disc, where disks normally reproduce data magnetically, whereas discs reproduce data optically with lasers. Combinations of the foregoing must also be included within the scope of computer-readable medium. Any processor and the storage medium can be housed in an ASIC. The ASIC can be housed in a user terminal. As an alternative, the processor and the storage medium can be housed as discrete components in a user terminal.

As used herein, the computer program products comprising computer-readable media include all forms of computer-readable medium except up to point where that medium is not considered as non-established transitory propagation signals.

The scope of the present invention is defined in the attached claims.

What is claimed is:

1. A system for the placement of dental implants, comprising:
one or more posts, each one of said one or more posts being configured for the coupling thereof in a dental implant of a patient, and each one of said one or more posts including a plurality of first locators made of a radiologically visible material;
an image acquisition system configured for acquiring at least one three-dimensional image;
a processing unit configured for processing the acquired at least one three-dimensional image; and
a reference element configured for the positioning thereof around the teeth of said patient, said reference element including a series of second locators made of a radiologically visible material, distributed at prefixed distance and position on a surface of the reference element;
wherein the image acquisition system is configured for acquiring the three-dimensional image of the one or more posts and of said reference element, when the one or more posts with their first locators are coupled in the implant and when the reference element with its second locators is positioned around the teeth of the patient; and
wherein the processing unit is configured for processing the acquired three-dimensional image by:
implementing a first algorithm which:
detects how the second locators in the reference element are arranged in the image,
calculates a distance between the second locators in the image, and
calculates a correction factor by comparing said calculated distance with said prefixed distance and position; and
implementing a second algorithm which:
detects in the image the first locators,
groups the detected first locators, in correspondence to each one of said one or more posts, by the number of elements and by proximity,
applies an adjustment factor, taking into account said calculated correction factor, to one or more of the groups using a third point iteration algorithm, providing a series of geometrical transformations of the first locators, and
prepares a file using said series of geometrical transformations, such that the placement and axial orientation are indicated for each one of said one or more posts.

2. The system according to claim 1, wherein the first locators made of a radiologically visible material comprise one or more radiopaque markers.

3. The system according to claim 1, wherein the one or more posts comprise a radiotransparent body which houses encapsulated therein said first locators.

4. The system according to claim 1, wherein the three-dimensional image is a tomographic image obtained using a cone-beam computed tomography or a computed axial tomography technique.

5. The system according to claim 1, wherein the reference element has a horseshoe shape configured for being positioned inside the mouth of the patient.

6. The system according to claim 1, wherein the reference element has a horseshoe shape configured for being secured outside the mouth of the patient.

7. The system according to claim 15, wherein the reference element is made of a plastic material.

8. The system according to claim 1, further comprising a control element having known dimensions and configured for the coupling thereof to the patient during image acquisition, the control element comprising a ruby ball.

9. A method for the placement of dental implants, comprising:
acquiring, by an image acquisition system, at least one three-dimensional image of first locators made of a radiologically visible material included in one or more posts and of a reference element when each of said one or more posts is coupled in a dental implant of a patient and when the reference element is positioned around the teeth of the patient, said reference element including a series of second locators made of a radiologically visible material, distributed at prefixed distance and position on a surface of the reference element; and
processing, by a processing unit, said at least one acquired three-dimensional image by:
implementing a first algorithm which:
detects how the second locators in the reference element are arranged in the image,
calculates a distance between the second locators in the image, and
calculates a correction factor by comparing said calculated distance with said prefixed distance and position; and
implementing a second algorithm which:
detects in the image the first locators,
groups the detected first locators, in correspondence to each one of said one or more posts, by the number of elements and by proximity,
applies an adjustment factor, taking into account said calculated correction factor, to one or more of the groups using a third point iteration algorithm, providing a series of geometrical transformations of the first locators, and
prepares a file using said series of geometrical transformations, such that the placement and axial orientation are indicated for each one of said one or more posts.

10. The method according to claim 9, wherein, before detecting the arrangement of the second locators in the reference element, the first algorithm checks whether or not the image has experienced movement during the acquisition by using a control object having known dimensions, which is coupled to the patient during the acquisition, and comparing characteristics of the acquired image with the control object, wherein if a result of said movement check certifies that the image has experienced movement, it is discarded.

11. The method according to claim 10, wherein the comparison comprises checking intensity characteristics, including brightness or grayscale level, and morphological characteristics, including diameter and sphericity, of the image with the control object.

12. The method according to claim 9, wherein the three-dimensional image is a tomographic image obtained using a cone-beam computed tomography or a computed axial tomography technique.

13. A non-transitory computer program product including code instructions which, once implemented in a computing system, cause a processing unit of the computing system to perform a processing of at least one acquired three-dimensional image by:
implementing a first algorithm which:
detects how second locators in a reference element are arranged in the image, said reference element being configured for the positioning thereof around the teeth of a patient and including a series of said second locators, the second locators being made of a radiologically visible material and being distributed at prefixed distance and position on a surface of the reference element,
calculates a distance between the second locators in the image, and
calculates a correction factor by comparing said calculated distance with said prefixed distance and position; and
implementing a second algorithm which:
detects in the image first locators, said first locators being included in one or more posts and being made of radiologically visible material, each one of said one or more posts being configured for the coupling thereof in a dental implant of a patient,
groups the detected first locators, in correspondence to each one of said one or more posts, by the number of elements and by proximity,
applies an adjustment factor, taking into account said calculated correction factor, to one or more of the groups using a third point iteration algorithm, providing a series of geometrical transformations of the first locators, and prepares a file using said series of geometrical transformations, such that the placement and axial orientation are indicated for each one of said one or more posts.
